(19) United States
(12) Patent Application Publication
(10) Pub. No.: US 2024/0293164 A1
(43) Pub. Date: Sep. 5, 2024

(54) METHODS AND COMPOSITIONS OF CRYOABLATION WITH DRUG DELIVERY

(71) Applicant: Theromics, Inc., West Bridgewater, MA (US)

(72) Inventors: William Keun Chan Park, Westerly, RI (US); Damian E. Dupuy, Osterville, MA (US)

(21) Appl. No.: 18/593,526

(22) Filed: Mar. 1, 2024

Related U.S. Application Data
(60) Provisional application No. 63/461,115, filed on Apr. 21, 2023.
(60) Provisional application No. 63/449,822, filed on Mar. 3, 2023.

(51) Int. Cl. A61B 18/02; A61B 18/00; A61K 9/06; A61K 38/26; A61K 38/28; A61K 45/06; A61K 47/42; A61K 47/64; A61K 49/00

(52) U.S. Cl. CPC A61B 18/0218; A61K 9/06; A61K 38/26; A61K 38/28; A61K 45/06; A61K 47/42; A61K 47/643; A61K 49/0073; A61B 2018/00577

(57) ABSTRACT

A method for cryoablation of a tissue of a patient includes positioning a cryoablation gel composition adjacent to a tissue of the patient, and cryoablating the tissue and cryoablation gel composition with a cryoprobe. The cryoablation gel composition includes a polymer carrier and a thermal accelerant bound to the polymer carrier. The cryoablation gel composition may include a therapeutic drug. Cryoablating the tissue and the cryoablation gel composition can achieve a lethal isotherm at temperatures between −30 and −50° C. in a shorter duration of time than when compared to cryoablating the tissue without the cryoablation gel composition. Cryoablating the tissue and cryoablation gel composition can achieve a larger treatment volume in the tissue than when compared to cryoablating the tissue without the cryoablation gel composition. Furthermore, cryoablating the tissue and cryoablation gel plus drug composition can release the drug to treat the cryoablated cells with the therapeutic drug.

### PRIORITY

This patent application claims priority from provisional U.S. patent application No. 63/449,822, filed Mar. 3, 2023, entitled, “CRYOABLATION METHODS AND COMPOSITIONS,” and naming William Keun Chan Park and Damian E. Dupuy as inventors; and this application also claims priority from provisional U.S. patent application No. 63/461,115, filed Apr. 21, 2023, entitled, “CRYOABLATION METHODS AND COMPOSITIONS,” and naming William Keun Chan Park and Damian E. Dupuy as inventors, the disclosures of both of which are incorporated herein, in its entireties, by reference.

### FIELD

Illustrative embodiments of the invention generally relate to cryoablation and, more particularly, various embodiments of the invention relate to enhancing cryoablation effectiveness by the use of a cryo-gel.

### BACKGROUND

Ablation is a treatment that destroys diseased tissue (e.g., cancers and/or tumors) without removing them. Ablation techniques can be used in patients with a few small tumors and when surgery is not a good option (often because of poor health or reduced liver function). Often, ablation can be done without surgery by inserting a needle or probe into the tumor through the skin. The needle or probe is guided into place with imaging techniques.

In ablation, special probes may be used to “burn” or “freeze” cancers. Computed Tomography (CT), Ultrasound (US), or Magnetic Resonance Imaging (MRI) may be used to guide and position the needle probe into the tumor. This requires only a tiny hole, usually less than 3 mm via into which the probe is introduced. When the probe is within the cancer, it is attached to a generator which “burns” or “freezes” the cancer. “Freezing” a cancer, referred to as cryoablation, can be achieved by use of a cryoprobe which can deliver temperatures substantially below 0° C.

### SUMMARY OF VARIOUS EMBODIMENTS

In accordance with one embodiment of the invention, a method for cryoablation of a tissue of a patient includes positioning a cryoablation gel composition adjacent to a tissue of the patient, and cryoablating the tissue and cryoablation gel composition with a cryoprobe. The cryoablation gel composition includes a thermal accelerant. Cryoablating the tissue and the cryoablation gel composition may achieve a lethal isotherm at temperatures between −30 and −40° C. in a shorter duration of time than when compared to cryoablating the tissue without the cryoablation gel composition. Cryoablating the tissue and cryoablation gel composition may achieve a larger treatment volume in the tissue than when compared to cryoablating the tissue without the cryoablation gel composition.

In some embodiments, the thermal accelerant may include a carrier including an albumin. The thermal accelerant may also include an ionic component. Further, the thermal accelerant may also include an imaging component. The polymer carrier may include at least one of a-PMMA, a-PS, or a-PVC.

In some embodiments, the cryoablation gel composition may undergo a phase change as the cryoablation gel composition components rearrange themselves in response to lowering the temperature of the cryoablation gel composition to less than 20° C. The phase change event may complete within about 5° C.

In some embodiments, the cryoablation of the tissue of the patient in the presence of the cryoablation gel composition may result in a faster temperature drop, a lower treatment temperature, and a larger ablation volume than cryoablation of the tissue of the patient without the cryoablation gel composition. The increase in a speed of the temperature drop, the decrease in the treatment temperature, and the increase of the cryoablation volume may on the volume of the cryoablation gel composition.

In some embodiments, positioning the cryoablation gel composition may include injecting the cryoablation gel composition into the tissue. The cryoablation gel composition may have a viscosity value of between about 500 cP and about 7,000 cP. The injected cryoablation gel composition may remain at the target site.

In some embodiments, the cryoablation gel composition may further include a drug. The drug may include a cytotoxic or immune modulatory anti-tumor agent. The cytotoxic anti-tumor agent may directly target FcRn over expressed tumor cells using pinocytosis of the albumin with the cytotoxic anti-tumor agent. The cytotoxic anti-tumor agent may include at least one of TLR-9 or STING agonists. The at least one of TLR-9 or STING agonists may utilize the immune surveillance APC cells comprising at least one of dendritic cells, lymphocytes (B and T cells), NK cells, or macrophages.

In accordance with another embodiment of the invention, a cryoablation gel composition includes a polymer and a thermal accelerant. The thermal accelerant includes an ionic component and an imaging component. The thermal accelerant may also include a carrier comprising an albumin.

In some embodiments, the cryoablation gel composition may include common contrast agents to aid determining the position at which the gel is injected, such as iohexol, gadovist or tantalum. These contrast agents may also be used as imaging components.

In some embodiments, the cryoablation gel composition may further include a drug configured to be associated with the carrier. The drug may be configured to be eluted out following exposure of the drug and the carrier to the cryoablation. The drug may be eluted out to mitigate a disease directly or play a role as a modulator of patients' immune responses while minimizing adverse effects.

In some embodiments, the polymer may include one or more of naturally occurring biopolymers, artificial biopolymers, or genetically modified biopolymers. The polymer may include ionic functionalities. The ionic functionalities may include one or more of amino (—NR₃⁺), carboxylate (—CO₂⁻), phosphate (P(O)O₃⁻), sulfonium (R₃S⁺), or other ionic groups.

In some embodiments, the ionic component may include at least one or more of alkaline metal ions, alkaline earth metal ions, or transition metal ions.

In some embodiments, the albumin may include human serum albumin (HSA). The HSA may serve as a drug delivery vehicle. The HSA drug delivery vehicle may deliver the drug via one or more of non-covalently binding, covalently binding, or genetic fusion strategies. The drug may further include one or more of Albiglutide, Semaglutide, Abraxane, or Levemir.

The thermal accelerant may further include an anti-tumor drug. The anti-tumor drug may include at least one of a STING agonist, a TLR9 agonist, or a cytotoxic agent.

In some embodiments, the cryoablation gel composition may further include a drug configured to be associated with the cryoablation gel composition. The drug may be configured to be eluted out following exposure of the drug and the cryoablation gel composition to the cryoablation. The drug may be associated with a cryoablation gel by affinity force or by being covalently tethered.

In accordance with another embodiment of the invention, a cryothermally-activated combined treatment composition includes a therapeutic agent, and a thermal accelerant. The thermal accelerant agent is configured to enhance ablation treatment, be impregnated with the therapeutic agent, and elute the therapeutic agent after exposure to cryogenic temperatures from a cryogenic source. The combined treatment composition is cryogenically activated by exposure to energy from the cryogenic source. The therapeutic agent may be associated with the thermal accelerant by at least one of protein binding or covalent bonding.

After the thermal accelerant is exposed to the cryoprobe, the thermal accelerant may be configured to solidify and becomes coupled with the ablated tissue. The thermal accelerant is configured to begin to elute a portion of therapeutic agent.

The thermal accelerant includes a carrier including an albumin, an ionic component including at least one chaotrope, and an imaging component.

The albumin may include human serum albumin or bovine serum albumin. The chaotrope may include at least one of calcium chloride, cesium chloride, lithium chloride, potassium chloride, rubidium chloride, sodium chloride, sodium citrate, trisodium citrate, sodium tryptophanate, citric acid, octanoic acid, or a combination thereof. The imaging component may include at least one of NaCl, CsCl, iohexol, or albumin.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically illustrates an experimental setup for performing an in-vitro experiment using an agarose phantom according to many of the embodiments described herein.

FIG. 1B shows a photograph of an experimental setup where cryoablation gel was added to agarose phantom according to many of the embodiments described herein.

FIG. 1C shows a temperature profile of the cryoablation gel shown and other test gels.

FIG. 1D shows a temperature profile of the cryoablation gel shown and another gel at different distances from the cryoablation gel.

FIG. 1E shows a temperature profile of the cryoablation gel and compares it to the temperature profiles of an agarose phantom media control at different distances from the cryoablation gel.

FIG. 2A shows a photograph of a top view of a bovine liver tissue at the 10th minute of cryoablation.

FIG. 2B shows a post-ablation (3 minutes following ablation) photograph of a side view of the cryoablated tissue after it was sectioned through the middle.

FIG. 3 shows a phase diagram for a NaCl+H₂O solution.

FIG. 4A shows thermal conductivity (left axis) and calorimetry (right axis) measurements as a function of temperature for NaCl+H₂O for different NaCl concentrations.

FIG. 4B shows differential scanning calorimetry measurements of a thermal gel to give heat flow as a function of temperature.

FIG. 4C shows differential scanning calorimetry measurements of pure water to give heat flow as a function of temperature.

FIG. 5 shows a plot of temperature dependent thermal conductivity (TC) in three polymers, a-PMMA, a-PS, and a-PVC.

FIG. 6A shows the chemical structure of a-PMMA.

FIG. 6B shows the chemical structure of a-PS.

FIG. 6C shows the chemical structure of a-PVC.

FIG. 7 shows the involvement of FcRn in cancer biology.

FIG. 8 illustrates therapeutic exploitation of FcRn upregulation in cancer.

FIG. 9 illustrates a combination therapy of cryoablation with cryoablation gel plus anti-tumor drugs according to many of the embodiments described herein.

FIG. 10 shows a schematic flow chart illustrating a method for cryoablation of a tissue of a patient according to many of the embodiments described herein.

FIG. 11 shows a schematic flow chart illustrating a method to treat a tissue with a cryoablation gel composition with cryoablation by a cryoablation probe according to many of the embodiments described herein.

### DESCRIPTION OF ILLUSTRATIVE EMBODIMENTS

Illustrative embodiments apply cryoablation of a tissue of a patient by positioning a cryoablation gel composition adjacent to a tissue of the patient, and then cryoablating the tissue and cryoablation gel composition with a cryoprobe. Moreover, some embodiments involve a cryoablation gel composition, and the preparation of such. Details of illustrative embodiments are discussed below.

### Cryoablation

Cryoablation is a procedure in which an extremely cold liquid or an instrument called a cryoprobe is used to freeze and destroy abnormal tissue. A cryoprobe is cooled with substances such as liquid nitrogen, liquid nitrous oxide, or compressed argon gas. Cryoablation may be used to treat certain types of cancer and some conditions that may become cancer.

Ablation occurs in tissue that has been frozen by at least one of three mechanisms:
1. formation of ice crystals within cells, thereby disrupting membranes, and interrupting cellular metabolism among other processes;
2. coagulation of blood, thereby interrupting blood flow to the tissue in turn causing ischemia and cell death; or
3. induction of apoptosis, the so-called programmed cell death cascade.

A common application of cryoablation is in its use to ablate solid tumors found in the lung, liver, breast, kidney and prostate. Although sometimes applied in cryosurgery through laparoscopic or open surgical approaches, most often cryoablation is performed percutaneously (through the skin and into the target tissue containing the tumor) by a medical specialist, such as an interventional radiologist.

### Thermodynamics of a Cryoprobe

Percutaneous cryoablation is performed by inserting cryoprobes into malignant tissue under imaging guidance. After placement of the cryoprobe, the cryoprobe is rapidly cooled, removing heat from the surrounding tissue by conduction through physical contact with the cryoprobe. Rapid cooling of the cryoprobe takes place by means of the Joule-Thompson effect, whereby rapid expansion of a gas that does no work (adiabatic expansion) results in a change in the temperature of the gas. The cryoprobe is essentially a high-pressure, closed-loop, gas expansion system. When high-pressure room temperature gas (typically argon) reaches the distal aspect of the cryoprobe, the argon is forced through a throttle (narrow opening) and then allowed to rapidly expand to atmospheric pressure. The rapid expansion of the argon causes a decrease in the temperature of the gas (the Joule-Thompson effect), which is rapidly transferred by convection and conduction to the metallic walls of the cryoprobe. The depressurized gas is vented back out of the hub of the needle. Warming of the cryoprobe and thawing of the tissue is performed through the same system using high-pressure helium, which warms the cryoprobe during expansion to atmospheric pressure.

### Transfer of Heat

The Joule-Thompson cooling effect causes temperatures near the Joule-Thompson port to approach that of liquid argon, or −187° C., but actual temperatures at the probe surface vary between-130° C. to −150° C. The extremely cold temperature at the probe surface is transferred to the tissue in contact with it and progresses at all directions from the active tip of the probe forming the isotherm layers over time.

### Lethal Isotherm

To destroy the target tumor cells by cryoablation, the temperature should be lowered <−30° C. Depending on the temperature, a cryoablation procedure often requires about 15 minutes or more. During this time, a typical progression of the “ice-forming”, e.g., 0° C. and lethal temperature is less than 2 cm and less than 1 cm from the probe, respectively, when agar phantom was used. Therefore, the lethal isotherm cryoablation of the tumor size >2 cm cannot be achieved by the approach under the same cryoablation conditions. This limitation can be potentially overcome by utilizing multiple cryoprobes, and a larger shaft diameter (e.g., 2.4 mm instead of 1.7 mm) can help improve the performance but only moderately. Another limiting factor to achieve the lethal isotherm is heat sink effect. A highly perfused organ such as the liver has a large number of blood vessels carrying blood at 36.5° C. in all directions, which makes cryoablation less effective at achieving the lethal isotherm (i.e., <−30° C.) at the periphery of the target tumor >2 cm in diameter.

### Cryoablation Gel

The cryoablation gel is a thermal accelerant comprising a human protein-based formulation with a high ionic content. The high ionic content composition of the cryoablation gel allows it to cool tissue faster than a control, to go to a lower treatment temperature than for a control, and to cool a larger volume of tissue than can be cooled in a control experiment. The thermal accelerant may be comprised of three components, 1) polymer (natural or artificial) as a carrier; 2) an ionic component or equivalent for overall charge and/or viscosity balance; and 3) an imaging component.

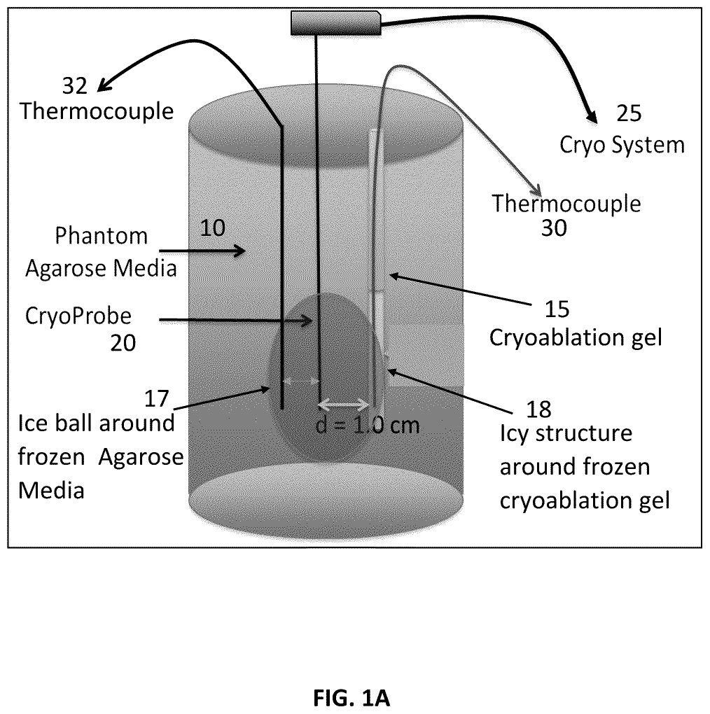

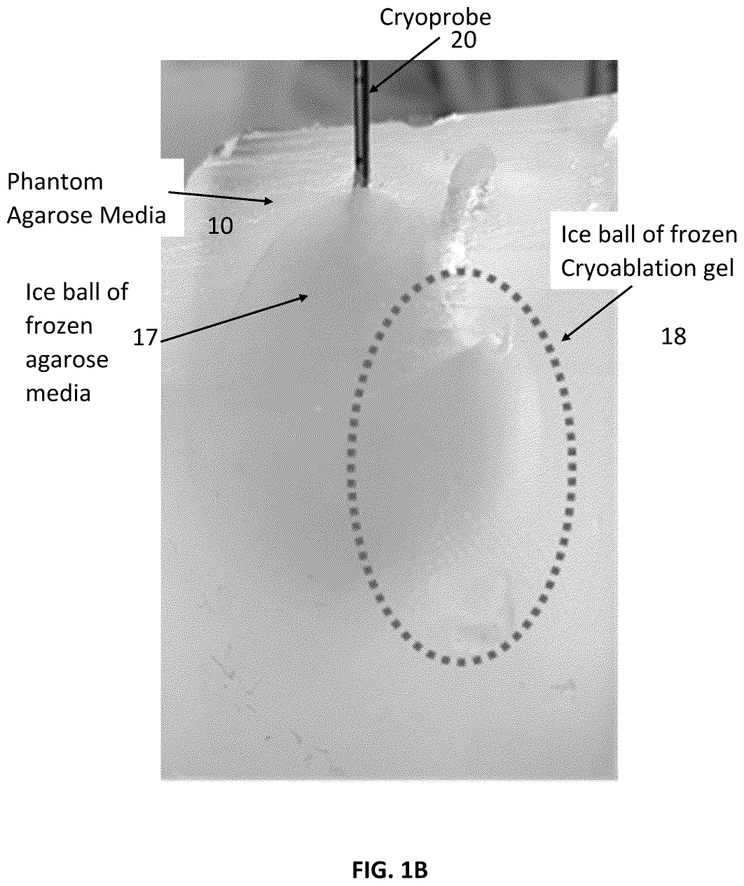

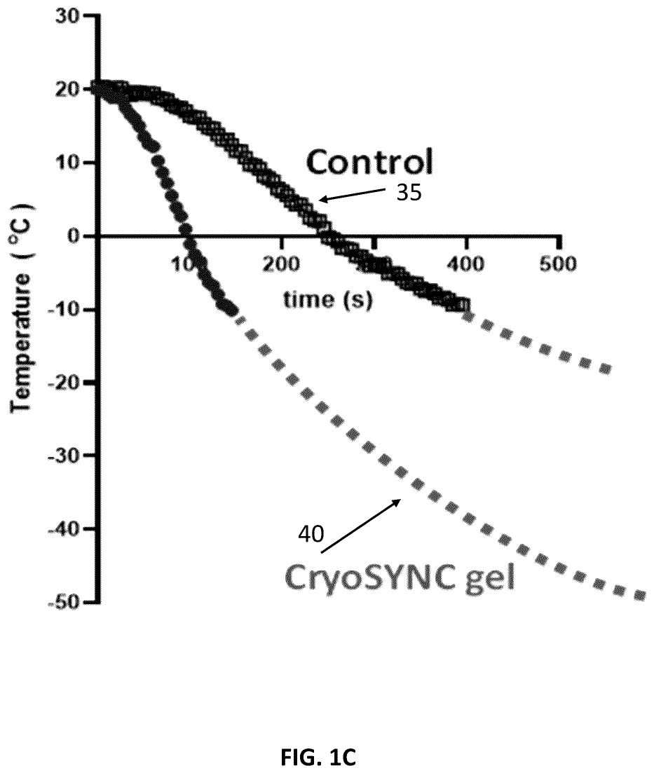

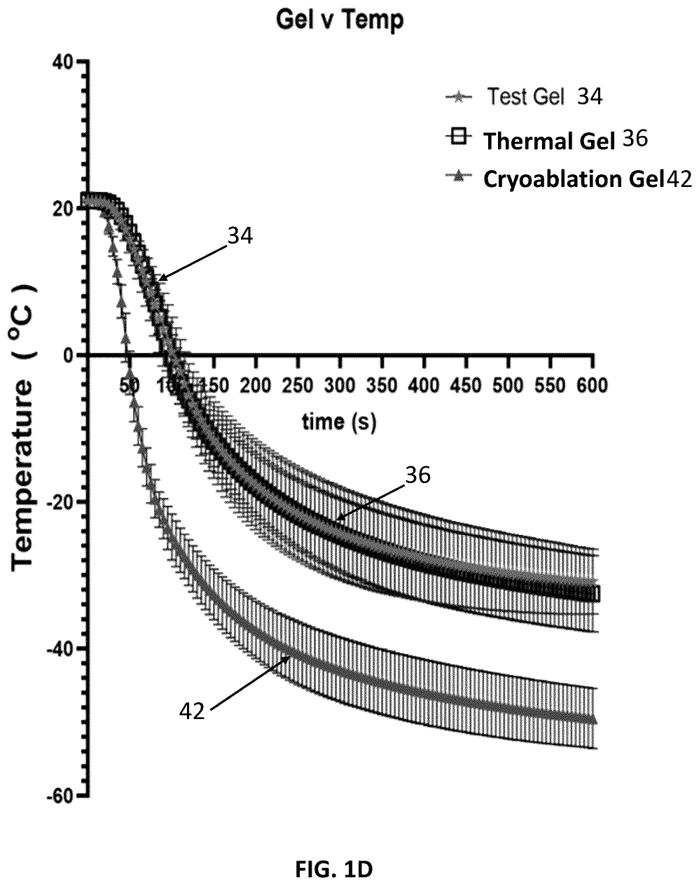

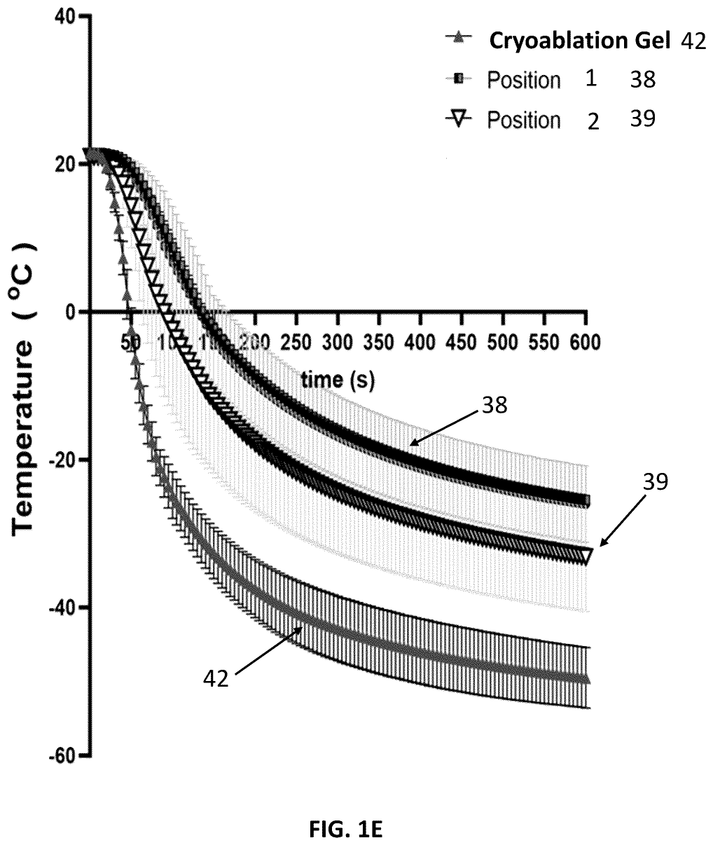

FIG. 1A schematically illustrates an experimental setup for performing cryoablation in an in-vitro experiment using an agarose phantom 10. Agarose phantoms are utilized in cryothermal experiments, particularly in the field of cryogenic thermal therapy and/or hypothermia research, because agarose gels can be engineered to mimic the thermal conductivity of human tissue. This property allows researchers to simulate how heat and cold propagates through different types of tissue, helping them understand the effects of cryoablation on target tissues and surrounding structures.

An agarose phantom media (e.g., agarose media, or agarose phantom) 10 was prepared using 1 w/v % agarose. A sample of cryoablation gel 15 was placed in the agarose phantom 10, and a thermocouple 30 was inserted into the cryoablation gel. A cryoprobe 20 (Endocare Cryosystem, cryoprobe PCS-17), as part of a cryosystem 25, was placed 5 cm deep from the top of the phantom media 10 and a 1 cm from the cryoablation gel and thermocouple 30. Another thermocouple 32 was inserted into the phantom agarose media 10 and was positioned 1 cm from the cryoprobe 20. (The schematic illustration in FIG. 1A is not drawn to scale.)

Upon of cooling of the cryoprobe 20, the agarose phantom media 10 freezes into an ice ball 17 of agarose phantom media. The cryoablation gel 15 also freezes into an icy, frozen structure 18 around the frozen cryoablation gel. Some agarose media 10 may also freeze around the frozen cryoablation gel 18.

FIG. 1B shows a photograph of an actual experimental setup where cryoablation gel 15 was added to the phantom agarose media 10. A cryoprobe 20 was inserted into the bag of phantom agarose media 10 and cryoablation gel 15. The cryoprobe was located 1 cm from the sample of cryoablation gel. Thermocouples (not shown) were inserted into the bag. One of the thermocouples was inserted into the cryoablation gel 15 at 1 cm from the cryoprobe 20, and the other thermocouple was inserted into the agarose media 10 at 1 cm from the cryoprobe.

The cryoprobe 20 was activated and cooled the mixture of agarose media 10 and cryoablation gel 15. The agarose media 10 and cryoablation gel 15 froze into an agarose media ice ball 17 and a cryoablation gel ice ball 18. The dashed ellipse 18 identifies the region where the cryoablation 15 gel was frozen into an icy structure (e.g., ice ball).

Temperature measurements of the agarose phantom (e.g., control) 10 were initiated 2 minutes after beginning the cryoablation and were continued for 10 minutes. The temperature profile 35 of the control agarose phantom is shown in FIG. 1C.

Temperature measurements of the cryoablation gel with the agarose phantom were also initiated 2 minutes after beginning the cryoablation and were continued for 10 minutes. The temperature profile 40 of the cryoablation gel in the control agarose phantom is also shown in FIG. 1C.

The cryoablation gel temperature fell more rapidly and to a lower temperature than the control about 2 cm away from the gel, as shown in FIG. 1C. The temperature profiles of both the cryoablation gel 40 and the control 35 are shown in FIG. 1C. The data shows that the temperature of cryoablation gel 40 rapidly falls below −40° C. within 5 minutes, while the data shows a much slower rate of the temperature decrease for the control 35 under the same conditions. The lowest temperature observed for the cryoablation gel sample in the experimental ablation duration of 10 minutes is approximately −50° C., while the lowest temperature observed for the control sample (agarose phantom without cryoablation gel) in the ablation duration of 10 minutes is approximately only −15° C. This data shows that adding cryoablation gel to the agarose phantom leads to a faster temperature drop and an ultimately lower temperature than agarose phantom without the cryoablation gel.

FIG. 1D shows temperature profiles from another experiment comparing the cryoablation gel 42 and two other thermal test gels 34, 36. The cryoablation gel 42 has a faster temperature drop and an ultimately lower temperature than the other thermal gels. The thermal test gels 34, 36 have lower pH levels than the cryoablation gel 42. The addition of trisodium citrate to the experimental thermal gels increased the pH (e.g., made it more basic) and increased the ionic conductivity of the cryoablation gel relative to the earlier thermal gels. The cryoablation gel 42 brings the temperature to lower than −35° C. within 2 minutes. This is significant because this temperature is considered to be tumoricidal. The temperature decreases below −35° C. and reaches below −50° C. while the other test gels did not reach −35° C. during the cryoablation (10 minutes).

FIG. 1E shows a temperature profile of the cryoablation gel and compares it to the temperature profiles of an agarose phantom media control at different distances from the cryoablation gel. This experiment illustrates the effect that the cooling cryoablation gel has on the surrounding media as a function of distance away from the cryoablation gel. The temperature profile 39 was measured at about 1 cm from the cryoablation gel, while the temperature profile 38 was measured at greater than 1.5 cm from the cryoablation gel. The salient feature here involves the fact that the temperature decease profile of the cryoablation gel 42 is significantly lower than any other locations without the gel, and that the thermal cooling effects of the cryoablation gel decreases with increasing distance away from the cryoablation gel.

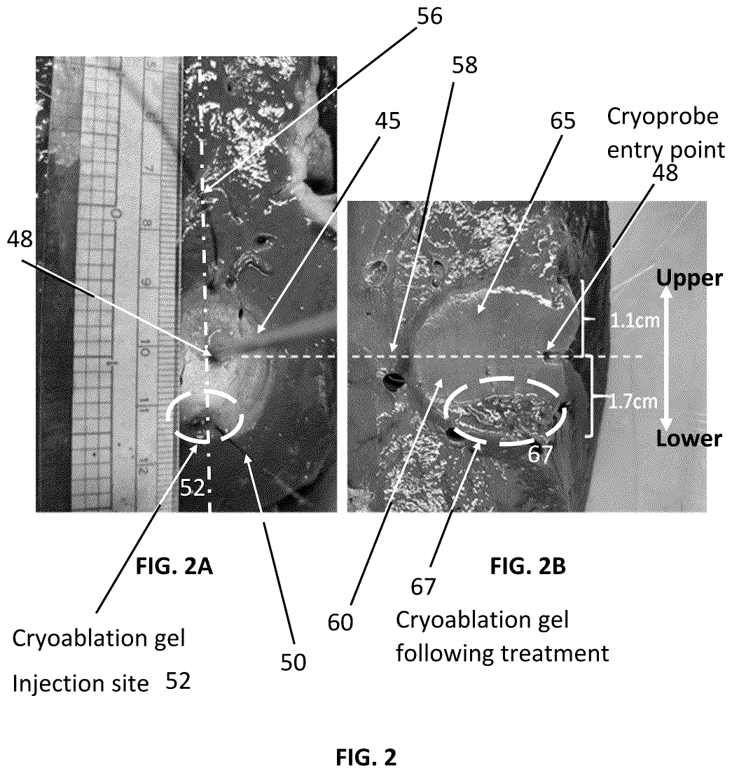

Ex vivo cryoablation experiments using bovine liver were performed. FIG. 2 shows photographs from an ex vivo cryoablation experiment using bovine liver. As shown in FIG. 2A, a cryoprobe 45 (Endocare, PSC-17) was placed in bovine liver tissue 4 cm deep from top of the liver surface at the cryoprobe 45 entry site 48. Cryoablation gel (0.7 mL) was injected at a site 52 to the same depth of the cryoprobe at 1 cm from the cryoprobe 45. A first thermocouple 50 was placed in the cryoablation gel at the cryoablation gel injection site 52. A second thermocouple 55 was counter laterally placed for the control (without the gel). Cryoablation was performed for 10 minutes. Similar temperature profiles were obtained with the bovine liver tissue and the control tissue as seen in the in vitro experiments described above.

FIG. 2A shows a photograph of a top view of the bovine liver tissue at the 10th minute of cryoablation.

FIG. 2B shows a post-ablation (3 minutes following ablation) photograph of the cryoablated tissue after it was sectioned through the middle along the dashed line 56. The dashed line 58 in FIG. 2B indicates a figurative dividing line created by the cryoprobe 45 that separates the lower portion 60 of the liver effected by the cryoprobe 45 from the upper portion 65 portion. The lower portion 60 had the cryoablation gel injected in it, while the upper portion 65 served as a control in that it did not have any cryoablation gel injected into it

Upon cooling by the cryoprobe 45, the temperature reached by the upper portion 65 was reduced to about −20° C. The radius of the affected area of the upper portion was about 1.1 cm.

Upon cooling by the cryoprobe 45, the temperature reached by the lower portion 60 was reduced to about −50° C. The liver tissue in the lower portion 60 that was in proximity to the cryoablation gel was expanded to be larger. The radius of the lower portion 60 expanded to a 1.7 cm radius, compared with the upper portion 65 of the liver tissue, with 1.1 cm that served as the control.

Also apparent in FIG. 2B is some of the cryoablation gel 67 following treatment (e.g., cryoablation experiment). The photograph of the cryoablation gel 67 shows that the cryoablation gel 67 aligns with the injection site 52 in FIG. 2A.

### Thermal Conductivity

Thermal conductivity is one of the key properties that are important in cryobiological applications.

$$q = - k \frac{T 2 - T 1}{L} \qquad Eq 1$$

As shown in Eq 1, heat flux q gives the rate per unit area, at which heat flows in a given direction. In many materials, q is observed to be proportional to the temperature difference (T2-T1) and inversely proportional to the separation distance L.

### Thermal Conductivity of Pure and Salt Water

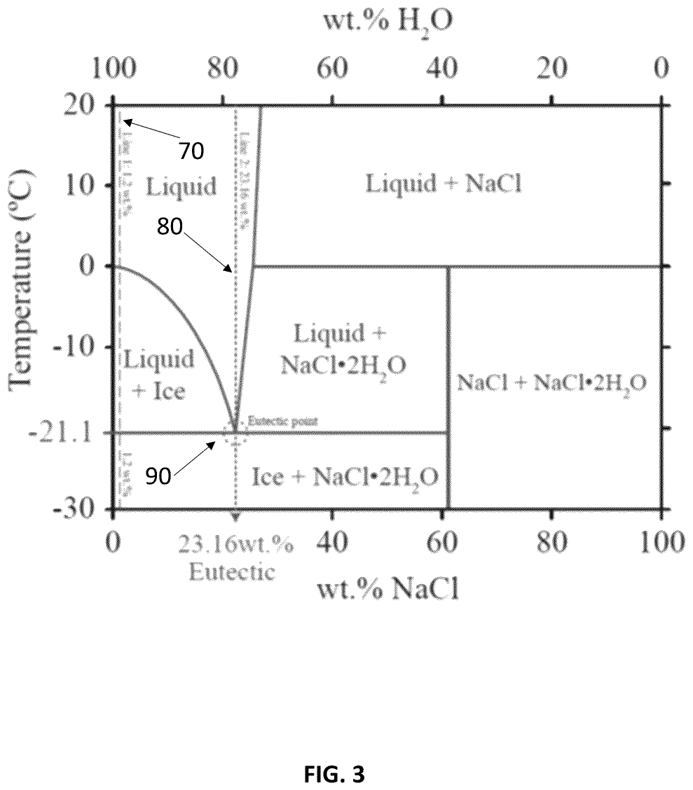

FIG. 3 shows a phase diagram for the NaCl+H₂O solution. From FIG. 3, Line 1 70: 1.2 wt. % describes a temperature change from 20° C. to −30° C. for dilute 1.2 wt. % NaCl+H₂O solution. Decreasing the temperature of the 1.2 wt. % NaCl+H₂O along the line 70 shows a solution starting from 20° C. that has no phase change in the solution until the temperature of the solution goes below the curved red solid line at slightly below 0° C. At this point (e.g., 0° C.), pure H₂O ice starts to develop in the solution and the concentration of the liquid (unfrozen solution) increases until the temperature reaches the eutectic temperature of −21.1° C. for NaCl+H₂O. As the temperature falls below −21.1° C., no more liquid will be found as it will be pure water ice and NaCl·2 H₂O crystals known as hydrohalite (61 wt. % NaCl). Another solution with a eutectic concentration of 23.16 wt. % of NaCl salt will follow Line 2 80 of FIG. 3, precipitation of both ice and NaCl·2 H₂O occurs at the −21.1° C. eutectic temperature.

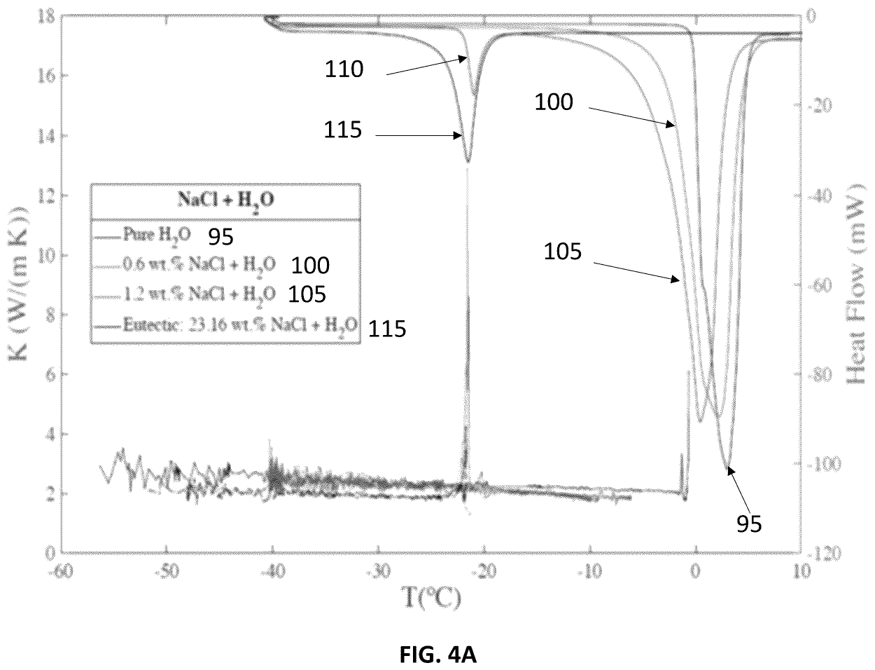

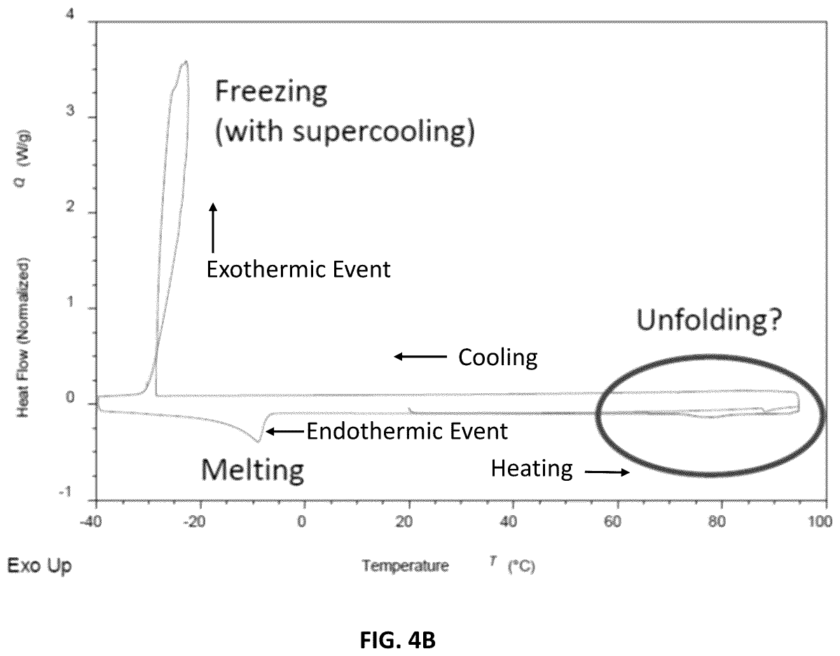

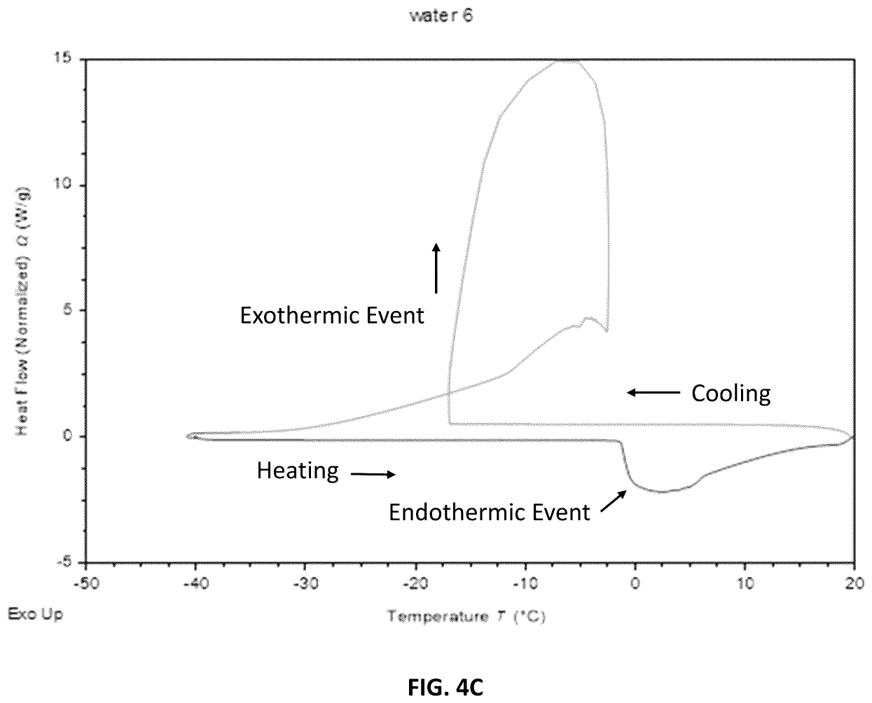

When thermal conductivity is measured for both pure water and salt water systems, it is apparent that the heat transfer from liquid water to ice is an exothermic process as shown in FIG. 4A. FIG. 4A shows thermal conductivity (left axis) and calorimetry (right axis) measurements as a function of temperature for NaCl+H₂O with different NaCl concentrations.

The blue trace 95 is pure H₂O (e.g., water), and it can be seen that the water has an exothermic process at about 4° C. That is, as the water begins to freeze into ice, it releases heat into the surrounding environment, thus the process is exothermic.

The pink trace 100 is 0.6 wt. % NaCl+H₂O, and it can be seen that the 0.6 wt. % NaCl+H₂O has an exothermic process at about 2° C.

The red trace 105 is 1.2 wt. % NaCl+H₂O, and it can be seen that the 1.2 wt. % NaCl+H₂O has an exothermic process at about 0° C. There is also a second endothermic event 110 for the 1.2 wt. % NaCl+H₂O at about −21° C. This exothermic event corresponds to the solidification of hydrohalite crystals of NaCl·2 H₂O, at a 61 weight percent of NaCl to H₂O.

The black trace 115 is a brine solution that is at the eutectic concentration of 23.16 wt. % NaCl+H₂O. For the eutectic brine solution (23.16 wt. % NaCl+H₂O), the phase-change occurs at −21.1° C., with a salient feature that it requires less energy to form the ice.

The phase-change temperature shifts to lower temperature as the NaCl content increases. This is due to the fact that the increasing concentration of NaCl in an aqueous solution lowers the freezing point of the mixture of salt and water in the solution.

There is also an exothermic event for the 110 and 115 traces at about −21° C. These events correlate to the rearrangement of the water molecules and NaCl in the salt solution to form hydrohalite crystals of NaCl·2 H₂O, at a 61 weight percent of NaCl to H₂O. The large exotherm at about −21° C. for the eutectic solution 115 compared to the more dilute 1.2% solution 110 indicates that the rearrangement of the H₂O and NaCl molecules (e.g., phase change) in the more concentrated solution released more energy into the surrounding to form the hydrohalite crystals of NaCl·2 H₂O than did the lower concentration solution.

FIG. 4B shows differential scanning calorimetry (DSC) measurements of the cryoablation gel to give heat flow as a function of temperature. The DSC scan in FIG. 4B shows that a sharp thermodynamic change (e.g., phase change) occurs in the cryoablation gel composition as the gel components rearrange themselves in response to the lowering temperatures at about −20° C.; and the phase changing event is complete within about 5° C. This is also an exothermic event. Upon heating, the cryoablation gel melts at about −10° C.

FIG. 4C shows differential scanning calorimetry measurements of water to give heat flow as a function of temperature. An exothermic event begins as the sample is cooled to about −18° C.; and the phase changing event is more than 10° C. wide. This process is more than 4×more exothermic than the crystallization of cryoablation gel, showing that the water crystallization process is much more chaotic with high entropy and takes a while to reach an ordered state. On the contrary, the cryoablation gel is a more highly ordered substance and the crystallization event produces less energy due to the relatively lower entropy.

### Thermal Conductivity of Non-Polar Polymers

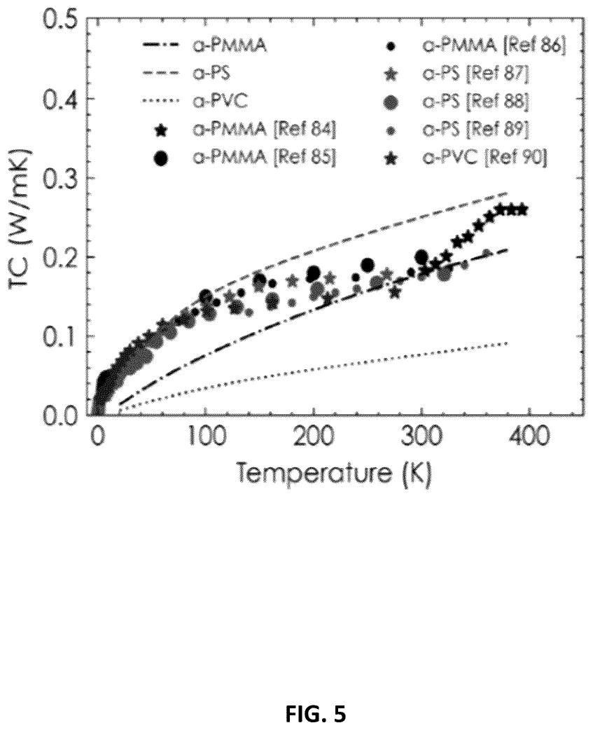

When non-polar organic polymer materials, and in particular, thermal plastics, are examined for thermal conductivity, these polymers reveal that their thermal conductivity values are small and furthermore, decrease as temperatures are lowered as shown in FIG. 5.

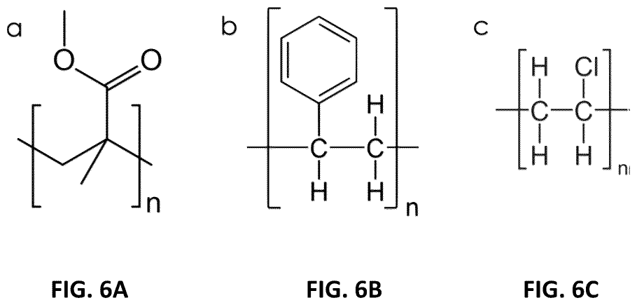

FIG. 5 shows a plot of temperature dependent thermal conductivity (TC) in three polymers, a-PMMA, a-PS, and a-PVC. The temperature dependence is calculated from Green Kubo Mode Analysis (GKMA) with quantum correction. Solid lines are calculated results, dots are experimental values reported in the literature. The chemical structures of thermoplastics (e.g., polymers) in the present study are shown in FIG. 6A-C: FIG. 6A corresponds to a-PMMA;

FIG. 6B corresponds to a-PS; and FIG. 6C corresponds to a-PVC.

### Thermal Diffusivity

Thermal Diffusivity is the thermal conductivity divided by density and specific heat capacity at constant pressure. It measures the rate of transfer of heat of a material from the hot end to the cold end.

$$α = \frac{k}{ϱ Cp} \qquad Eq 2$$

where
k is thermal conductivity (W/(m·K))
C_{ρ} is specific heat capacity (J/(kg·K))
ρ is density (kg/m³)

There are no organic materials with higher thermal conductivity than ice: ca. 2 W/(mK) at 0° C. The approximate diffusivity values of ice and saline ice are shown below in Table 1.

TABLE 1
The parameters affecting thermal diffusivity for ice and saline ice.

| | | Thermal | Specific | Thermal |
|---|---|---|---|---|
| Temperature | Density | Conductivity | Heat | Diffusivity |
| T | ρ | k | Cₚ | α 10⁻⁷ |
| (° C.) | (kg/m³) | (W/mK) | (kJ/kgK) | (m²/s) |
| (Water) | 999.8 | | | |
| 0 | 916.2 | 2.22 | 2.050 | 11.8 |
| −10 | 918.9 | 2.30 | 2.000 | 12.5 |
| −40 | 920.8 | 2.63 | 1.818 | 15.7 |
| −80 | 924.1 | 3.19 | 1.536 | 22.4 |
| −100 | 925.7 | 3.48 | 1.389 | 27.1 |
| −10.06 | ~1,005 | ~2 | 3.108*⁹ | ~6.4 |

*The Cp value comes from the 6 wt. % NaCl solution (ice)

For pure ice, thermal conductivity gradually increases as temperature decreases. The increase in the diffusivity values are influenced by the decrease of specific heat (asymptote towards ˜1.3 (kJ/kg K)) as the temperature is lowered. For saline ice (e.g., 6 wt % NaCl solution (ice)), the trends are similar to the pure ice with the decrease of specific heat towards 2.33 KJ/kg K influencing the diffusivity value approximately one half of the pure water. The trends of thermal conductivity and diffusivity of pure water are very similar to those of the blood plasma in the temperature range (−10 to −100° C.) as shown in Table 2, below at the end of this document. This is because of the high water content (91-99%) in blood plasma even if the plasma salinity (0.9 wt. %) is taken to consideration.

### Cryoablation Gel as a Thermal Accelerant

As mentioned above, thermal diffusivity measures the rate of heat transfer of a material from the hot end to the cold end. That is, the larger the thermal diffusivity of the material, the better efficiency of the material as a thermal accelerant in making the surrounding regions colder during cryoablation. That is, thermal accelerants include materials that accelerate thermal cooling effects and improve the efficacy in cryothermal treatments to facilitate rapid cooling of tissues.

As shown in Table 2, the blood plasma has higher conductivity and diffusivity than any other biological materials or tissue listed. For example, the thermal diffusivity of blood plasma increases from 9.7×10−7 m2/s at −10° C. to 26.9×10−7 m2/s at −100° C. as the thermal conductivity changes from 2.03 to 3.19 W/mK, respectively showing that the change in thermal diffusivity is greater than the change in thermal conductivity. This means that the specific heat of the plasma decreases significantly over the range of temperatures (−10 to −100° C.). More significantly, the decrease of the specific heat of plasma is more dramatic in the temperature region, i.e., 0 to −10° C.

As FIG. 1C shows, cryoablation gel, a formulation that consists of a concentrated mixture of polymer(s) with ionic functionalities and their counter ions in aqueous solution, shows a far more efficient temperature decrease in comparison to the control. It is assumed that the rapid decrease in temperature of the gel originates from the precipitous drop of the specific heat values in the low sub-zero temperature range (0 to −10° C.) than that of pure ice (the control): For example, 6 wt. % NaCl solution has the specific heat value of 111.9 (KJ/kgK) at −1° C. as compared to 3.1 (KJ/kgK) at −10° C. This dramatic change of the specific heat values of the gel can affect the change in the thermal diffusivity values during the early cryoablation (36.5 to −10° C.). Important thermal properties of a cryoablation gel include: density, specific heat, thermal conductivity thermal diffusivity, and enthalpy. In some embodiments, the thermal accelerant is a preparation of a serum albumin or other albumin, as described further below, together with certain electrolytes that condition its viscosity, or thermal accelerant properties, and preferably also provide imaging under one or more medical imaging modalities such as MRI, ultrasound or x-ray CT imaging. Using CT for image guidance, the desired amount of the thermal accelerant with known CsCl concentration can be added to the cryoablation gel and deposited in the boundary of the tumor mass. Subsequently, upon cooling, the injected cryoablation gel freezes into a solid of predetermined ablation shape and volume. The cryoablation gel is then cooled by the cryoprobe to reach tumoricidal temperature (<−40° C.) in the targeted area.

Cryoablation gel may be mixed with drugs to be injected for cryoablation. Post ablation, the injected drugs can be eluted out to mitigate the disease directly or play a role as a modulator of patients' immune responses while minimizing adverse effects.

Another property of the cryoablation gel is viscosity. The cryoablation gel composition has a viscosity value of between about 500 cP and about 7,000 cP. Viscosity values of between about 500 cP and about 7,000 cP aid the gel stay at the target site of tissue once injected.

The polymers that can be used to make the cryoablation gel include the naturally occurring biopolymers: e.g., protein polymers, DNA, RNA, nucleosides, carbohydrates, lipids, glycoproteins and metallo-proteins. The polymers can also include the artificial or genetically modified biopolymers, such as dendrimer or various polymers in nanoparticle forms. The polymers herein contain ionic functionalities, e.g., amino (—NR₃), carboxylate (—CO₂⁻), phosphate (P(O)O₃⁻), sulfonium (R₃S⁺) and other ionic groups. The polymers may include either natural or artificial, for example, albumins, silk, wool, chitosan, alginate, pectin, DNA, cellulose, polysialic acids, dendritic polylysine, poly(lactic-co-glycolic) acid (PLGA), gellan, polysaccharides and poly-aspartic acid, and combinations thereof.

The ionic component can include counter ions that can be used in the cryoablation gel are opposite to the charge present in the polymer to make the overall charge neutral: any alkaline metal ions such as Na⁺, K⁺, Cs⁺ alkaline earth metal ions such as Ca⁺⁺, Mg⁺⁺, and transition metal ions of Mn, Fe, Co, Cu, and Zn may be included. For the polymer cations like amino and sulfonium ions, halide ions (F⁻, Cl⁻, Br⁻, I⁻), carboxylate (—CO₂⁻), phosphate (P(O)O₃⁻) ions can be the counter ions. In some embodiments, the thermal accelerant includes a chaotrope. The chaotrope may be configured to adjust charge distribution within the polymer. Chaotropic salts or solutions may be used as part of tissue preparation or as part of the cryoablation process to enhance the efficacy of other agents, such as therapeutic agents (e.g., drugs). These substances can help to disrupt the structure of cellular membranes or proteins, making tissues more susceptible to cryoablation.

The cryoablation gel may include trisodium citrate which has the chemical formula of Na₃C₆H₅O₇. Trisodium citrate may be referred to simply as “sodium citrate”, though sodium citrate can refer to any of the three sodium salts of citric acid. Trisodium citrate dihydrate is a tribasic salt of citric acid. The addition of trisodium citrate raises the pH of the cryoablation gel to about a pH of 8, and can make the pH of the cryoablation gel to be between 7.5 and 8.5. The trisodium citrate also increases the ionic concentration of the cryoablation gel, so that it has a great ionic conductivity. The trisodium citrate can also function as a therapy agent (e.g., drug). That is, the trisodium citrate may contribute to the cryoablation gel as a therapy drug, in addition to performing as a stabilizer, and/or an ionic component.

The cryoablation gel may also include may caprylic acid, also known under the systematic name octanoic acid or C8 acid. Caprylic acid is a straight-chain saturated fatty acid and a medium-chain fatty acid. It is a conjugate acid of an octanoate.

Trisodium citrate and caprylic acid may be included in the cryoablation gel as ionic components, and they may each be added as stabilizers, or as a combination of uses within the gel.

The cryoablation gel may include sodium tryptophanate and/or tryptophan as a stabilizer. Tryptophan contains an α-amino group, an α-carboxylic acid group, and a side chain indole, making it a polar molecule with a non-polar aromatic beta carbon substituent.

The cryoablation gel may include benzoic acid which is an organic compound which is described by the chemical formula C₆H₅COOH.

An imaging component can also increase the thermal performance of the cryoablation gel. For example, iohexol and/or tantalum can be included in the cryoablation gel to help x-ray or CT scan diagnosis or location finding. The iohexol and/or tantalum may also act in the cryoablation gel to achieve temperatures of less than (<)−40° C. within 3 minutes. (see slide 15). Furthermore, the cryoablation gel with imaging component (e.g., iohexol or tantalum) can enable achievement of tumoricidal temperatures below (<)−35° C. during cryoablation. At temperatures below −30° C. during cryoablation, it can be difficult to visualize a tumor since it may appear as a big ice ball. However, the addition of one or more the imaging agents to the cryoablation gel can make the tumor visible with a CT, in addition to the temperature effect of the imaging agent.

The aqueous medium may be a buffer solution such as phosphate, citrate-based buffers at desired pH of the overall solution.

The concentration of the polymer in cryoablation gel may be greater than 50 g/L to have the desired viscosity (>10 cP).

Table 2 below is a list of thermal properties of biological materials and tissue in the cryogenic regime.

TABLE 2
List of thermal properties of biological materials and tissue in the cryogenic regime.
Thermal properties of logic biologically relevant liquids and chemicals in the subzero temperature domain*

| | | | Thermal | | | | Latent | |
|---|---|---|---|---|---|---|---|---|
| | Thermal | | Diffusivity × | | Specific | | Heat | |
| | Conductivity | | 10⁷ | | Heat | | (ice) | |
| Material | [W/mK] | | [m²/s] | | [J/gK] | | [J/g] | Reference |
| Blood | | | | | | | | [96] |
| packed cells | 1.24 | (−10° C.) | 6.8 | (−10° C.) | | | | |
| | 1.51 | (−40° C.) | 11.0 | (−40° C.) | | | | |
| | 1.98 | (−80° C.) | 17.2 | (−80° C.) | | | | |
| | 2.26 | (−100° C.) | 20.4 | (−100° C.) | | | | |
| plasma | 2.03 | (−10° C.) | 9.7 | (−10° C.) | | | | |
| | 2.31 | (−40° C.) | 15.1 | (−40° C.) | | | | |
| | 2.86 | (−80° C.) | 22.9 | (−80° C.) | | | | |
| | 3.19 | (−100° C.) | 26.9 | (−100° C.) | | | | |
| Whole | 1.64 | (−10° C.) | 8.7 | (−10° C.) | | | | |
| | 1.92 | (−40° C.) | 13.6 | (−40° C.) | | | | |
| | 2.38 | (−80° C.) | 20.4 | (−80° C.) | | | | |
| | 2.66 | (−100° C.) | 23.7 | (−100° C.) | | | | |
| DMSO (dimethyl sulphoxide) | | | | | | | | [97] |
| 100% | 0.2 | (−20° C.) | | | | | | |
| 75% (v/v) | 0.243 | (−20° C.) | | | | | | |
| 50% (v/v) | 0.318 | (−20° C.) | | | | | | |
| Ethanol | | | | | | | | [92] |
| 50% (w/w) | | | | | | | 55.3 | |
| 45% (w/w) | | | | | | | 58.3 | |
| 40% (w/w) | | | | | | | 65.7 | |
| Ethylene glycol | | | | | | | | [48] |
| 100% | 0.242 | (−5-−20° C.) | 0.934 | (−5-−20° C.) | | | | |
| 45% (w/w) | | | | | | | 44.9 | [91] |
| 40% (w/w) | | | | | | | 65.3 | |
| 27% (w/w) + glycerol (18%) | | | | | | | 53.3 | |
| Glycerol | | | | | | | | [97] |
| 100% | 0.258 | (−20° C.) | | | | | | |
| 75% (v/v) | 0.299 | (−20° C.) | | | | | | |
| 50% (v/v) | 0.362 | (−20° C.) | | | | | | |
| 50% (w/w) | | | | | | | 50.6 | [92] |
| 40% (w/w) | | | | | | | 86.8 | |
| 40% (w/w) + ethanol (10%) | | | | | | | 48.9 | |
| 30% (w/w) + ethanol (20%) | | | | | | | 39.9 | |
| 20% (w/w) + ethanol (30%) | | | | | | | 41.9 | |
| 10% (w/w) + ethanol (40%) | | | | | | | 38.9 | |
| | | | | | | | | [20] |
| 6M + 1 × PBS | 0.82 | (−28° C.) | | | 2.984 | (−20° C.) | 47.1 | |
| | 1.27 | (−65° C.) | | | 1.996 | (−73° C.) | | |
| | 1.05 | (−108° C.) | | | 1.114 | (−110° C.) | | |
| | 0.97 | (−147° C.) | | | 0.834 | (−148° C.) | | |
| 2M + 1 × PBS | 1.61 | (−27° C.) | | | 3.781 | (−5° C.) | 178.6 | |
| | 1.96 | (−64° C.) | | | 1.712 | (−73° C.) | | |
| | 2.15 | (−108° C.) | | | 1.206 | (−110° C.) | | |
| | 2.25 | (−147° C.) | | | 0.903 | (−148° C.) | | |
| 1M + 1 × PBS | 1.95 | (−28° C.) | | | 3.969 | (0° C.) | 238.2 | |
| | 2.33 | (−64° C.) | | | 1.641 | (−73° C.) | | |
| | 2.83 | (−109° C.) | | | 1.216 | (−110° C.) | | |
| | 3.11 | (−146° C.) | | | 0.959 | (−148° C.) | | |
| Glycol | | | | | | | | [97] |
| 100% | 0.253 | (−20° C.) | | | | | | |
| 46.4% (w/w) | 0.43 | (0° C.) | | | | | | |
| | 0.441 | (−10° C.) | | | | | | |
| | 0.437 | (−20° C.) | | | | | | |
| | 0.439 | (−30° C.) | | | | | | |
| HP5 (organ perfusate) | | | | | | | | [98] |
| whole | 1.962 | (−10.3° C.) | | | | | | |
| | 2.394 | (−30° C.) | | | | | | |
| | 2.556 | (−50° C.) | | | | | | |
| | 2.936 | (−70° C.) | | | | | | |
| +2M glycerol | 0.896 | (−10.3° C.) | | | | | | |
| | 1.221 | (−30° C.) | | | | | | |
| | 1.486 | (−50° C.) | | | | | | |
| | 1.65 | (−70° C.) | | | | | | |
| Milk (whole) | | | | | | | 289.8 | [68] |
| Propyl alcohol | | | | | | | | [99] |
| 35% (w/w) | | | | | | | 144.1 | |
| Propylene glycol | | | | | | | | [97] |
| 100% | 0.193 | (−20° C.) | | | | | | |
| 75% (v/v) | 0.286 | (−20° C.) | | | | | | |
| 50% (v/v) | 0.36 | (−20° C.) | | | | | | |
| 40% (w/w) | | | | | | | 57 | [100] |
| 30% (w/w) | | | | | | | 93.8 | [99] |
| 20% (w/w) | | | | | | | 145.7 | |
| 10% (w/w) | | | | | | | 214.4 | |
| 28% (w/w) + ethanol (7%) | | | | | | | 65 | |
| 36% (w/w) + glycerol (9%) | | | | | | | 26.1 | |
| 27% (w/w) + glycerol (18%) | | | | | | | 50.3 | |
| 14% (w/w) | | | | | | | 92.5 | |
| +propyl alcohol (21%) | | | | | | | | |

*weight and volume ratios are with respect to H₂O unless indicated otherwise.

Thermal properties of biological tissues and materials in the subzero temperature domain

| | | | Themal | | | | Latent | |
|---|---|---|---|---|---|---|---|---|
| | Thermal | | Diffusivity × | | Specific | | Heat | |
| | Conductivity | | 10⁷ | | Heat | | (ice) | |
| Material | [W/mK] | | [m²/s] | | [J/gK] | | [J/g] | Ref. |
| Beef | | | | | | | | |
| bovine fat | 0.193 | (+0.1° C.)^{a} | 0.59 | (+0.1° C.)^{a} | 2.3 | (−40° C.)^{c} | | [48]^{a} |
| | 0.266 | (−5° C.)^{a} | 0.98 | (−5° C.)^{a} | 1.55 | (−80° C.)^{a} | | [57]^{b} |
| | 0.216 | (−7.6° C.)^{b} | 1.54 | (−18° C.)^{a} | 1.00 | (−120° C.)^{a} | | [101]^{c} |
| | 0.3 | (−9.4° C.)^{b} | | | 0.85 | (−160° C.)^{a} | | |
| | 0.28 | (−18° C.)^{a} | | | | | | |
| bovine fat (74.5% water, | 0.478 | (0° C.) | 1.36 | (−20° C.) | | | | [102] |
| perpendicular to fiber) | 0.930 | (−5° C.) | | | | | | |
| | 1.20 | (−10° C.) | | | | | | |
| | 1.43 | (−20° C.) | | | | | | |
| bovine meat | 0.417 | (−7.3° C.)^{a} | | | 3.977 | (+4° C.)^{b} | 205.8 | [57]^{a} |
| | 0.448 | (−7.5° C.)^{a} | | | 3.68 | (−22° C.)^{c} | −256.2^{d} | [69]^{b} |
| | | | | | 2.0 | (−23° C.)^{b} | | [101]^{c} |
| | | | | | 1.884 | (−29° C.)^{b} | | [68]^{d} |
| | | | | | 1.842 | (−34° C.)^{b} | | |
| | | | | | 2.43 | (−34° C.)^{c} | | |
| | | | | | 1.8 | (−40° C.)^{b} | | |
| | | | | | 1.88 | (−57° C.)^{c} | | |
| | | | | | 1.63 | (−87° C.)^{c} | | |
| | | | | | 1.05 | (−169° C.)^{c} | | |
| bovine meat (74.5% water) | | | | | 3.6 | (0° C.) | | [103] |
| | | | | | 2.5 | (−20° C.) | | |
| | | | | | 2.1 | (−30° C.) | | |
| | | | | | 1.9 | (−40° C.) | | |
| bovine meat (75% water, 0.9% | 0.480 | (0° C.) | | | | | | [104] |
| fat, parallel to fiber) | 1.36 | (−10° C.) | | | | | | |
| | 1.54 | (−25° C.) | | | | | | |
| bovine meat (78.5% water) | | | | | | | | [102] |
| | | | | | 3.81 | (0° C.) | | |
| | | | | | 3.31 | (−20° C.) | | |
| | | | | | 2.89 | (−30° C.) | | |
| bovine meat, lean | | | | | | | | [102] |
| | 0.480 | (0° C.) | 4.72 | (−20° C.) | | | | |
| | 1.06 | (−5° C.) | | | | | | |
| | 1.35 | (−10° C.) | | | | | | |
| bovine meat, lean, neck | 1.57 | (−20° C.) | | | | | | [46] |
| bovine muscle | 0.415 | (−7° C.) | | | | | | [48] |
| | 0.425 | (+0.1° C.) | 1.05 | (+0.1° C.) | | | | |
| | 1.393 | (−5° C.) | 5.37 | (−5° C.) | | | | |
| eye loin, 69% water aged | 1.076 | (−18° C.) | 6.84 | (−18° C.) | | | | [105] |
| | 0.294 | (0° C.) | | | | | | |
| | 0.990 | (−3° C.) | | | | | | |
| | 1.04 | (−10° C.) | | | | | | |
| flank, lean, 3.4% water | 1.07 | (−17° C.) | | | | | | [104] |
| | 1.07 | (−10° C.) | | | | | | |
| round, 76% H2O, 3% fat | 1.21 | (−25° C.) | | | | | | [50] |
| | 0.47 | (+2° C.) | | | | | | |
| | 0.88 | (−4° C.) | | | | | | |
| udder, 9% H2O, 89% fat | 1.12 | (−13° C.) | | | | | | [104] |
| | 0.21 | (+2° C.) | | | | | | |
| | 0.29 | (−7° C.) | | | | | | |
| | 0.29 | (−12° C.) | | | | | | |
| Bone (femur, cow) | 0.33 | (−19° C.) | | | | | | [106] |
| Chicken | | | | | 4.354 | (−1.8° C.) | | [68] |
| | | | | | 3.3 | (−3.8° C.) | | |
| Duck | | | | | 3.056 | (−1.8° C.) | | [68] |
| | | | | | 2.47 | (−3.8° C.) | | |
| Eggs | | | | | | | 222.6 | [68] |
| Fish | 0.43 | (0° C.)^{a} | | | | | 205.8 | [107]^{a} |
| | 1.22 | (−10° C.)^{a} | | | | | −285.6^{b} | [68]^{b} |
| | 1.37 | (−20° C.)^{a} | | | | | | |
| sea trout | | | | | 3.684 | (+4° C.) | | [69] |
| | | | | | 2.135 | (−23° C.) | | |
| | | | | | 1.926 | (−29° C.) | | |
| | | | | | 1.8 | (−34° C.) | | |
| | | | | | 1.758 | (−40° C.) | | |
| Fruits | | | | | | | 252 | [68] |
| | | | | | | | −306.6 | |
| Gelatin | | | | | | | | [104] |
| 20% | 1.57 | (−10° C.) | | | | | | |
| | 1.56 | (−30° C.) | | | | | | |
| 12% | 1.89 | (−10° C.) | | | | | | |
| | 2.08 | (−30° C.) | | | | | | |
| 6% | 2.09 | (−10° C.) | | | | | | |
| | 2.28 | (−30° C.) | | | | | | |
| Kidney | | | | | | | | |
| 79.8% water | | | | | 4.1 | (0° C.) | | [108] |
| | | | | | 2.14 | (−23° C.) | | |
| | | | | | 1.63 | (−40° C.) | | |
| bovine, cortex | 0.454 | (+0.1° C.) | 1.18 | (+0.1° C.) | | | | [48] |
| | 1.535 | (−5° C.) | 4.71 | (−5° C.) | | | | |
| | 1.372 | (−18° C.) | 6.84 | (−18° C.) | | | | |
| Pork | | | | | | | 117.6 | [68] |
| | | | | | | | −138.6 | |
| ham | | | | | 3.475 | (+4° C.) | | [69] |
| | | | | | 1.884 | (−23° C.) | | |
| | | | | | 1.717 | (−29° C.) | | |
| | | | | | 1.633 | (−34° C.) | | |
| | | | | | 1.633 | (−40° C.) | | |
| pig fat | 0.360 | (−9.1° C.)^{a} | | | 1.6 | (−40° C.)^{b} | | [57]^{a} |
| | 0.366 | (−10° C.)^{a} | | | 1.21 | (−80° C.)^{b} | | [101]^{b} |
| | | | | | 1.03 | (−120° C.)^{b} | | |
| | | | | | 0.88 | (−160° C.)^{b} | | |
| pig fat (3.1% water) | 0.186 | (0° C.) | | | | | | [102] |
| | 0.227 | (−5° C.) | | | | | | |
| | 0.254 | (−10° C.) | | | | | | |
| | 0.291 | (−20° C.) | | | | | | |
| pig, lean, 6.1% fat, 72% water, | 1.43 | (−10° C.) | | | | | | [104] |
| parallel to fiber | 1.61 | (−25° C.) | | | | | | |
| pig, lean, 6.1% fat, 72% water, | 1.23 | (−10° C.) | | | | | | [104] |
| parallel to fiber | 1.38 | (−25° C.) | | | | | | |
| pig, lean, 76.8% water, | 0.478 | (0° C.) | 3.89 | (−20° C.) | | | | [102] |
| perpendicular to fiber | 0.767 | (−5° C.) | | | | | | |
| | 0.900 | (−10° C.) | | | | | | |
| pig, lean, neck | 1.29 | (−20° C.) | | | | | | [57] |
| | 0.783 | (−8° C.) | | | | | | |
| | 0.835 | (−8.4° C.) | | | | | | |
| pork, exterior, | 0.408 | (−9° C.) | | | | | | [104] |
| 6% H2O, 93% fat | 0.21 | | | | | | | |
| pork, leg, | | (+3-−24° C.) | | | | | | [50] |
| 75.9% H2O, 6.7% fat | 0.49 | (+6° C.) | | | | | | |
| | 1.28 | (−8° C.) | | | | | | |

Mediated Drug Delivery Using Cryoablation with Cryoablation Gel+ Drug

In some embodiments, a therapeutic agent (e.g., drug) may be combined with the cryoablation gel. That is, by using a combined cryoablation gel and drug composition, the effectiveness of cryoablation is increased and a therapeutic drug is delivered directly to the target tissue during cryoablation, and is eluted at the target tissue for days after ablation.

The drug or combination of drugs may include:
Alkylating Agents
   Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin
   Platinum Coordination Complexes
      Carboplatin, Cisplatin, Oxaliplatin
Antibiotics, Cytotoxic
   Bleomycin, Dactinomycin, Daunorubicin, Doxorubicin, Epirubicin, Idarubicin, Mitomycin, Mitoxantrone, Plicamycin, Valrubicin
Antimetabolites
   Antifolates: Methotrexate, Pemetrexed, Pralatrexate, Trimetrexate
   Purine Analogues: Azathioprine, Cladribine, Fludarabine, Mercaptopurine, Thioguanine
   Pyrimidine Analogues: Azacitidine, Capecitabine, Cytarabine, Decitabine, Floxuridine, Fluorouracil, Gemcitabine, Trifluridine/Tipracil
Biologic Response Modifiers
   Aldesleukin (IL-2), Denileukin Diftitox, Interferon Gamma
Histone Deacetylase Inhibitors
   Belinostat, Panobinostat, Romidepsin, Vorinostat
Hormonal Agents
   Antiandrogens: Abiraterone, Apalutamide, Bicalutamide, Cyproterone, Enzalutamide, Flutamide, Nilutamide
   Antiestrogens (including Aromatase Inhibitors): Anastrozole, Exemestane, Fulvestrant, Letrozole, Raloxifene, Tamoxifen, Toremifene
   Gonadotropin Releasing Hormone Analogues: Degarelix, Goserelin, Histrelin, Leuprolide, Triptorelin
   Peptide Hormones: Lanreotide, Octreotide, Pasireotide
Monoclonal Antibodies
   Alemtuzumab, Atezolizumab, Avelumab, Bevacizumab, Blinatumomab, Brentuximab, Cemiplimab, Cetuximab, Daratumumab, Dinutuximab, Dostarlimab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Teclistamab, Tositumom ab, Trastuzumab, Tremelimumab
Protein Kinase Inhibitors
   Abemaciclib, Acalabrutinib, Afatinib, Alectinib, Alpelisib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pemigatinib, Pexidartinib, Ponatinib, Regorafenib, Ribocicib, Rucaparib, Ruxolitinib, Selumetinib, Sonidegib, Sorafenib, Sunitinib, Talazoparib, Trametinib, Vandetanib, Vemurafenib, Vismodegib, Zanubrutinib
Taxanes
   Cabazitaxel, Docetaxel, Paclitaxel
Topoisomerase Inhibitors
   Etoposide, Irinotecan, Teniposide, Topotecan
Vinca Alkaloids
   Vinblastine, Vincristine, Vinorelbine
Miscellaneous
   Asparaginase (Pegaspargase), Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax

Human Serum Albumin (HSA) broadly serves as an excellent drug delivery vehicle via non-covalently binding, covalently binding, and genetic fusion strategies. For example, HSA-based or HSA-binding drugs such as Albiglutide, Semaglutide, Abraxane and Levemir have been successfully developed and clinically applied.

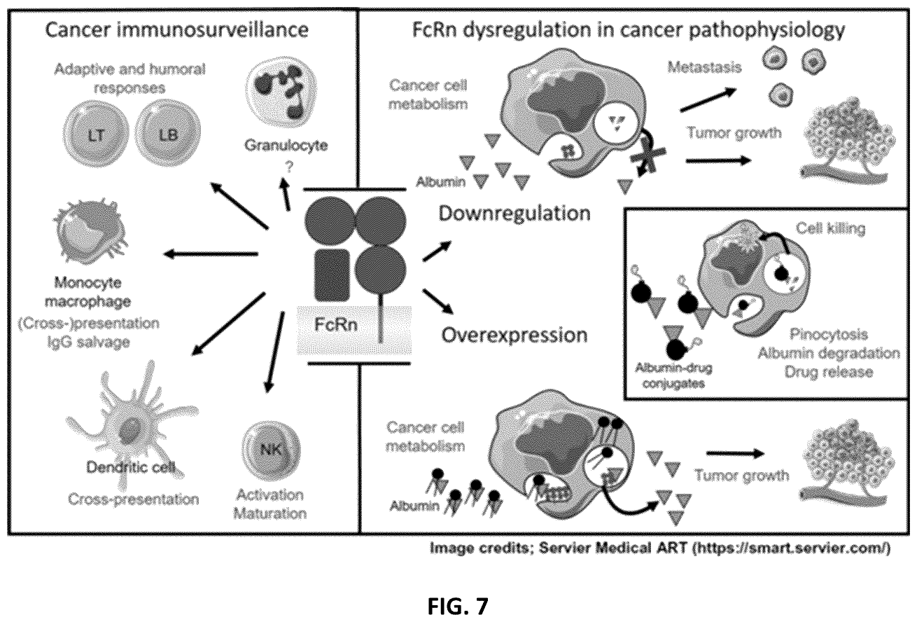

Since the neonatal IgG Fc receptor (FcRn) was discovered, it was found to be involved in immunoglobulin recycling and biodistribution, immune complexes routing, antigen presentation, humoral immune response, and cancer immunosurveillance. FIG. 7 shows the involvement of FcRn in cancer biology, and FcRn plays a part in cancer pathophysiology. In various types of cancers, such as lung and colorectal cancer, FcRn has been described as an early marker for prognosis. Dysregulation of FcRn expression by cancer cells allows them to increase their metabolism, and this mechanism can be exploited for passive targeting of cytotoxic drugs. However, the roles of this receptor depend on whether the studied cell population is the tumor tissue or the infiltrating cells, bringing forward the need for further studies.

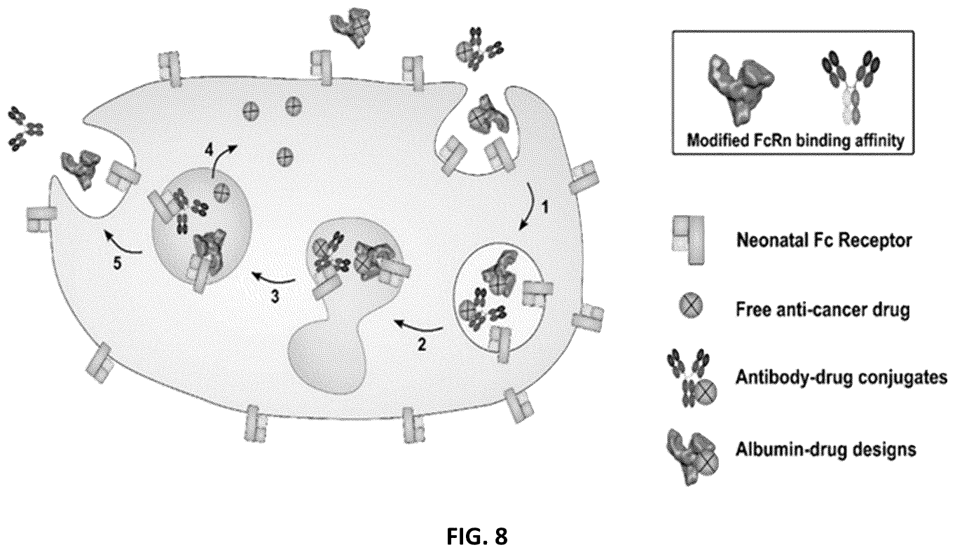

FIG. 8 illustrates therapeutic exploitation of FcRn upregulation in cancer. Enhanced endosomal uptake of antibody-drug conjugates or albumin-drug designs due to FcRn upregulation in solid tumors. Uptake of antibody-drug conjugates or albumin-drug designs. Endosomal sorting. Stimuli-responsive drug release and diffusion. Antibody and/or albumin is recycled to the cell surface. Insert depicts albumin and antibody variants with tunable FcRn affinities to modulate the uptake and cellular recycling.

FcRn (Neonatal Fc Receptor) is expressed in antigen-presenting cells (APCs) such as dendritic cells (DCs), macrophages, and B cells. The high-level expression of FcRn in these cells enables the specific intracellular trafficking of IgG and antigen-containing IgG immune complexes through the endolysosomal system.

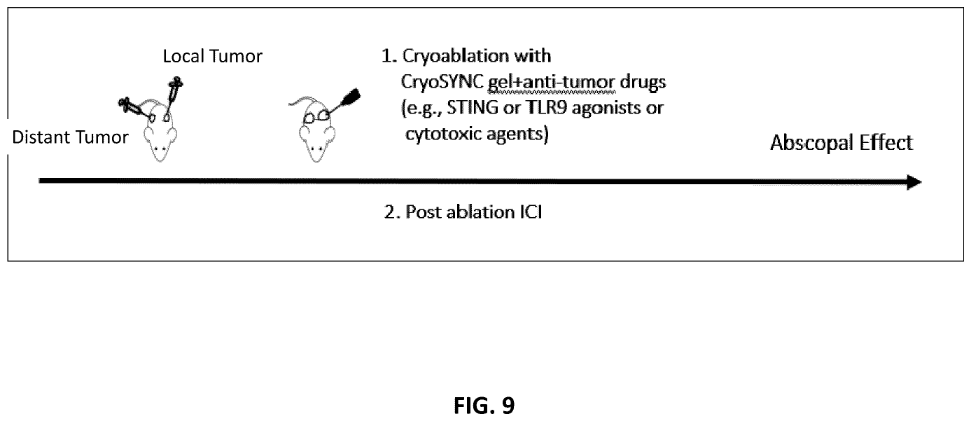

Based on the mechanisms of FcRn associated with HSA and IgG, a combination therapy may be employed, as shown in FIG. 9. FIG. 9 illustrates a combination therapy of cryoablation with cryoablation gel plus anti-tumor drugs according to an embodiment of the current disclosure. Anti-tumor agents directly target FcRn over expressed tumor cells using pinocytosis of albumin with the drug. TLR-9 or STING agonists utilize the immune surveillance APC cells such as dendritic cells, lymphocytes (B and T cells), NK cells, and macrophages, such that all express the FcRn receptors.

STING (stimulator of interferon genes) is an endoplasmic reticulum transmembrane protein that plays a central role in innate immunity against infection and cancer. Activation of STING mediates a multifaceted type-I interferon (IFN-I) response that promotes the maturation and migration of dendritic cells (DCs), and primes cytotoxic T lymphocytes and natural killer (NK) cells for spontaneous immune responses. STING agonists enhance the migration and killing of NK cells, improving therapeutic activity in patient-derived organotypic tumor spheroids.

Toll-like receptor 9 (TLR9) is a pattern recognition receptor that is predominantly located intracellularly in immune cells, including dendritic cells, macrophages, natural killer cells, and other antigen-presenting cells (APC). The primary ligands for TLR9 receptors are unmethylated cytidine phosphate guanosine (CpG) oligodinucleotides (ODN). TLR9 agonists induce inflammatory processes that result in the enhanced uptake and killing of microorganisms and cancer cells, as well as the generation of adaptive immune responses.

To restate, the cryoablation gel may be comprised of three components, 1) polymer (natural or artificial) as a carrier; 2) an ionic component or equivalent for overall charge and/or viscosity balance; and 3) an imaging component. They may also include stabilizers. The polymers may include either natural or artificial, for example, albumins, silk, wool, chitosan, alginate, pectin, DNA, cellulose, polysialic acids, dendritic polylysine, poly(lactic-co-glycolic) acid (PLGA), gellan, polysaccharides and poly-aspartic acid, and combinations thereof. The ionic component may include, M+X− or M2+Y2− (as a generalized formula Mn+Yn−), where M belongs to alkaline or alkaline earth metal such as Li, Na, K, Rb, Cs and X represents halides, acetate, and other equivalent counter balance to M+, and Y can be X2 or mixed halides, acetates, carbonate, sulfate, phosphate and other equivalent counter balance to M2+ as well as formic acid, glycolic acid, lactic acid, propionic acid, caproic acid, oxalic acid, malic acid, citric acid, benzoic acid, uric acid and their corresponding conjugate bases. Other organic components can independently be substituted as described in Wang, S. et al, Mol. Pharmaceutics 2015, 12, 4478-4487.

For imaging, including CT imaging (e.g., computed tomography), cesium, tantalum, iopamidol, iohexol, ioxilan, iopromide, iodixanol, ioxaglate, diatrizoate, metrizoate, iothalamate, ethiodized polymers such as PLGA, PEG, albumins, DNA, RNA, ionic poly-carbohydrates and the combinations there of can be utilized. Additional CT contrast agents that may be included in the cryoablation gel may include ioversol, iodixanol, iopromide, or iopamidol.

Additional contrast agents that may be used include gadoterate, gadobutrol, gadopentetate, gadobenate, or gadoteridol. These may include MR contrast agents for MR imaging (e.g., magnetic resonance imaging).

Albumin itself can be a US imaging (e.g., ultrasound) contrast enhancer by forming microbubbles. For ultrasound imaging, polymers are in general hypoechoic.

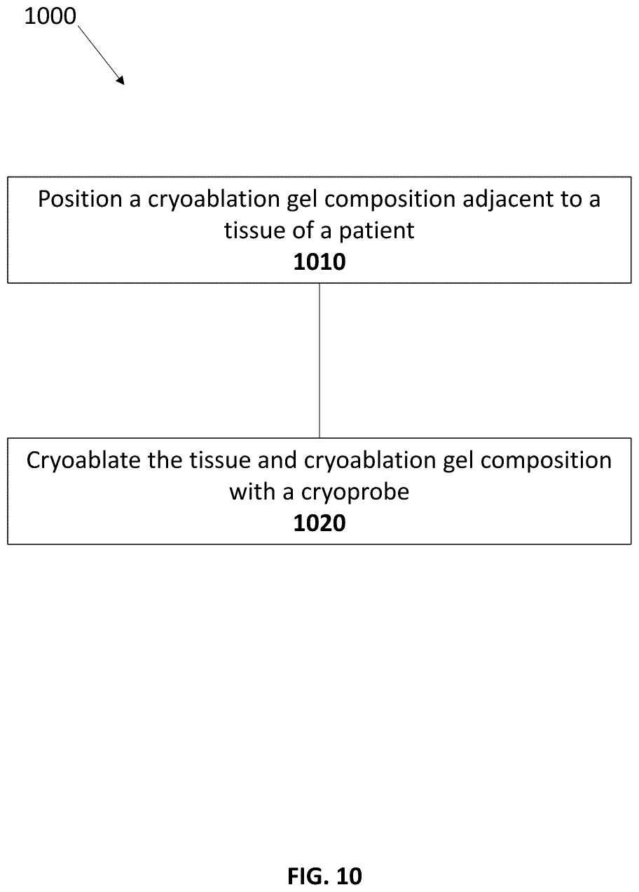

FIG. 10 shows a schematic flow 1000 chart illustrating a method for cryoablation of a tissue of a patient. At step 1010, a cryoablation gel composition is positioned adjacent to a tissue of a patient. The tissue is the target of the cryoablation therapy, and the cryoablation gel composition is positioned either adjacent to the tissue, or is injected into the tissue, or both. That is, in embodiments, the cryogenic gel may be placed on or around the tissue, or it may be injected into the tissue, or it may be positioned around and injected into the tissue.

At step 1020, the tissue and are cryoablated with a cryoprobe. The cryoprobe is inserted into the tissue and the cryoablation gel composition, and then the cryoprobe is operated to perform the cryoablation. The cryoprobe can rapidly cool to temperatures at or below −10° C. In experiments with the cryoablation gel composition, agarose phantom along with the cryoablation gel composition have reached temperatures below −40° C.

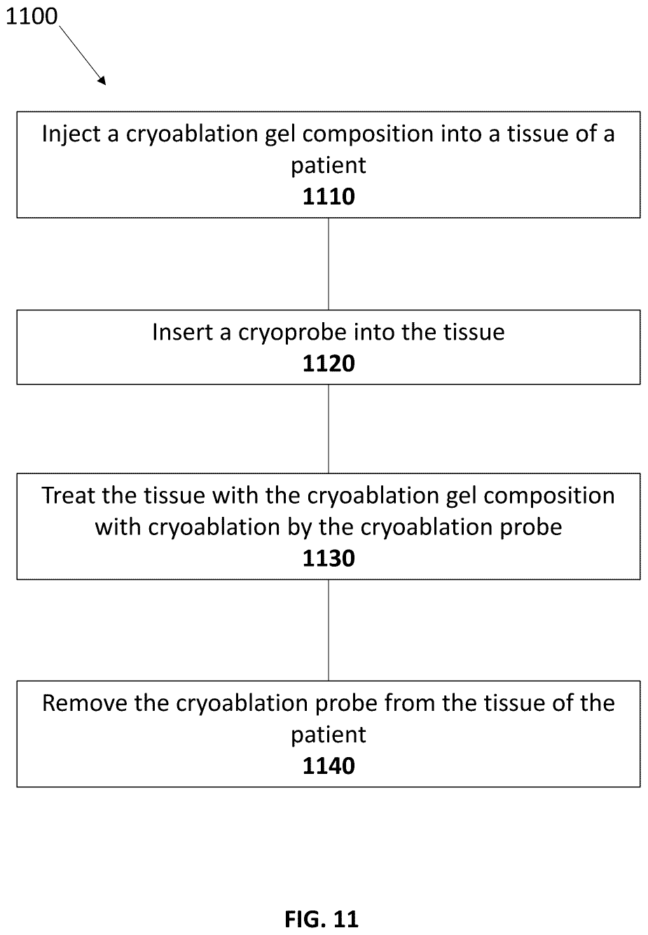

FIG. 11 shows a schematic flow chart 1100 illustrating a method to treat a tissue with a cryoablation gel composition with cryoablation by a cryoprobe. At step, 1110, a cryoablation gel composition is injected into a tissue of a patient. The injection of the cryoablation gel composition into the tissue may comprise injecting the cryoablation gel composition to be adjacent or around the tissue. The tissue is the target of the cryoablation therapy, and the cryoablation gel composition is positioned either adjacent to the tissue, or is injected into the tissue, or both. That is, in embodiments, the cryogenic gel may be placed on or around the tissue, or it may be injected into the tissue, or it may be positioned around and injected into the tissue.

At step 1120, the cryoprobe is inserted into the tissue. Imaging may be used to guide the insertion of the cryoprobe to the targeted region of the tissue. Alternatively, the is inserted into the tissue without the use of imaging. Computed Tomography (CT), Ultrasound (US), or Magnetic Resonance Imaging (MRI) may be used to guide and position the cryoprobe into the tumor. This requires only a tiny hole, usually less than 3 mm via into which the cryoprobe is introduced.

At step 1130, the tissue is treated with the cryoablation gel composition with cryoablation by the cryoprobe. When the cryoprobe is within the cancer, it is attached to a generator which “burns” or “freezes” the cancer. “Freezing” a cancer, referred to as cryoablation, can be achieved by use of a cryoprobe which can deliver temperatures substantially below 0° C.

At step 1140, the cryoablation probe is removed from the tissue of the patient. At the conclusion of the cryoablation treatment, the cryoprobe.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method for cryoablation of a tissue of a patient, comprising:
positioning a cryoablation gel composition adjacent to a tissue of the patient; and
cryoablating the tissue and cryoablation gel composition with a cryoprobe,
the cryoablation gel composition comprising a thermal accelerant.

2. The method of claim 1, wherein the thermal accelerant comprises:
a carrier comprising an albumin;
an ionic component; and
an imaging component.

3. The method of claim 1, wherein:
the cryoablation gel composition undergoes a phase change as the cryoablation gel composition components rearrange themselves in response to lowering the temperature of the cryoablation gel composition to less than −20° C.; and
the phase change event is complete within about 5° C.

4. The method of claim 1, wherein positioning the cryoablation gel composition comprises injecting the cryoablation gel composition into the tissue.

5. The method of claim 1, wherein the cryoablation gel composition has a viscosity value of between about 500 cP and about 7,000 cP.

6. The method of claim 5, wherein the injected cryoablation gel composition remains at the target site.

7. The method of claim 2, wherein the cryoablation gel composition further comprises a drug.

8. The method of claim 7, wherein the drug comprises a cytotoxic or immune modulatory anti-tumor agent.

9. The method of claim 8, wherein the cytotoxic anti-tumor agent directly targets FcRn over expressed tumor cells using pinocytosis of the albumin with the cytotoxic anti-tumor agent.

10. The method of claim 9, wherein the anti-tumor agent comprises at least one of TLR-9 or STING agonists.

11. The method of claim 10, wherein:
the at least one of TLR-9 or STING agonists utilize the immune surveillance APC cells comprising at least one of dendritic cells, lymphocytes (B and T cells), NK cells, or macrophages.

12. A cryoablation gel composition, comprising:
a polymer; and
a thermal accelerant comprising an ionic component and an imaging component.

13. The composition of claim 12, wherein the thermal accelerant further comprises:
a carrier comprising an albumin.

14. The composition of claim 13, further comprising:
a drug configured to be associated with the carrier, the drug being configured to be eluted out following exposure of the drug and the carrier to a cryoablation.

15. The composition of claim 14, wherein the drug is eluted out to mitigate a disease directly or play a role as a modulator of patients' immune responses while minimizing adverse effects.

16. The composition of claim 12, wherein the polymer comprises one or more of naturally occurring biopolymers, artificial biopolymers, or genetically modified biopolymers.

17. The composition of claim 16, wherein the polymer comprises:
ionic functionalities; and
the ionic functionalities comprise one or more of amino (—NR₃⁺), carboxylate (—CO₂⁻), phosphate (P(O)O₃⁻), sulfonium (R₃S⁺), or other ionic groups.

18. The composition of claim 12, wherein the ionic component comprises at least one or more of alkaline metal ions, alkaline earth metal ions, or transition metal ions.

19. The composition of claim 13, wherein the albumin comprises human serum albumin (HSA).

20. The composition of claim 19, wherein the HSA serves as a drug delivery vehicle.

21. The composition of claim 20, wherein the HSA drug delivery vehicle delivers the drug via one or more of non-covalently binding, covalently binding, or genetic fusion strategies.

22. The composition of claim 21, wherein the drug comprises one or more of Albiglutide, Semaglutide, Abraxane, or Levemir.

23. The composition of claim 12, wherein the thermal accelerant further comprises an anti-tumor drug.

24. The composition of claim 23, wherein the anti-tumor drug comprises at least one of a STING agonist, a TLR9 agonist, or a cytotoxic agent.

25. The composition of claim 12, further comprising:
a drug configured to be associated with the cryoablation gel composition, the drug being configured to be eluted out following exposure of the drug and the cryoablation gel composition to the cryoablation; and
the drug is associated with cryoablation gel composition by affinity force or by being covalently tethered.

26. A cryothermally-activated combined treatment composition, comprising:
a therapeutic agent; and
a thermal accelerant configured to:
enhance ablation treatment;
be impregnated with the therapeutic agent; and
elute the therapeutic agent after exposure to cryogenic temperatures from a cryogenic source, wherein the combined treatment composition is cryogenically activated by exposure to energy from the cryogenic source.

27. The composition of claim 26, wherein the therapeutic agent is associated with the thermal accelerant by at least one of protein binding or covalent bonding.

28. The composition of claim 26, wherein, after the thermal accelerant is exposed to the cryoprobe:
the thermal accelerant is configured to solidify and becomes coupled with the ablated tissue; and
the thermal accelerant is configured to begin to elute a portion of therapeutic agent.

29. The composition of claim 26, wherein:
the thermal accelerant comprises:
a carrier comprising an albumin;
an ionic component comprising at least one chaotrope; and
an imaging component.

30. The composition of claim 29, wherein:
the albumin comprises human serum albumin or bovine serum albumin;
the chaotrope comprises at least one of calcium chloride, cesium chloride, lithium chloride, potassium chloride, rubidium chloride, sodium chloride, sodium citrate, trisodium citrate, sodium tryptophanate, citric acid, octanoic acid, or a combination thereof; and
the imaging component comprises at least one of NaCl, CsCl, iohexol, or albumin.
